(19) United States
(12) Patent Application Publication
(10) Pub. No.: US 2003/0180278 A1
(43) Pub. Date: Sep. 25, 2003

(54) Use of a combination of active substances containing bioquinones for the production of cosmetic or dermatological preparations

(76) Inventors: Udo Hoppe, Heidmuhlen (DE); Weiping Mei, Hamburg (DE); Gerhard Sauermann, Wiemersdorf (DE)

Correspondence Address: NIXON & VANDERHYE, PC, 1100 N GLEBE ROAD, 8TH FLOOR, ARLINGTON, VA, 22201-4714, US

(21) Appl. No.: 10/343,118

(22) PCT Filed: Jul. 27, 2001
(86) PCT No.: PCT/EP01/08728

(51) Int. Cl. A61K 38/43; A61K 31/525; A61K 31/513; A61K 31/473; A61K 31/202; A61K 31/198; A61K 31/19; A61K 31/12

(52) U.S. Cl. 424/94.1; 514/284; 514/269; 514/560; 514/690; 514/251; 514/565; 514/574

(57) ABSTRACT

The invention relates to the use of a compound or several compounds from the group of bioquinones a) in combination with a compound or several compounds from the group of potassium channel openers and/or b) in combination with a compound or several compounds from the group of 5-alpha-reductase inhibitors, for the production of cosmetic or dermatological preparations for treatment of the scalp and for hair in order to prolong the anagenic phase and/or for the treatment and prophylaxis of seborrhoeic symptoms, optionally by additionally using one or several compounds from the group formed from carnitine, arginine, succinic acid, folic acid, conjugated fatty acids and respectively the derivatives thereof, in addition to antioxidants.

The subject of the invention is the use of a combination of active substances containing bioquinones for producing cosmetic or dermatological preparations, which brings about a prolongation of the anagenic phase of the hair growth cycle, or is also effective against dandruff.

It is known that hair growth corresponds to a cycle. Papillary hair is the name given to hair in the growth period, which is also known as the anagenic phase or anagen phase. In this phase the hair is anchored with its papilla in the skin. Approximately 80% of head hair is in the anagen phase for approximately 3 to 5 years. In a subsequent transitional phase (catagen phase) the hair migrates for approximately 2 weeks to the epidermis and remains there for approximately 3 to 4 months in a resting stage (telogen phase) until it finally falls out.

Hair loss over and above the norm is generally regarded as a serious cosmetic disorder, as are other hair growth disorders. Many agents have therefore already been proposed for the treatment of hair loss and balding, as well as hair growth agents which are intended to achieve or encourage hair growth.

From the German patent 12 96 310 and the PCT specification WO 85/04577 substances are also already known, which bring about a prolongation of the growth period, or prolongation of the anagen phase. This involves a substituted amino acid or substituted pyridyl pyrimidine.

From the U.S. Pat. No. 4,654,373 the topical application of the compound coenzyme Q₁₀ for the prevention of dystrophic or dysmetabolic conditions of the skin or its appendages is also known.

In the PCT specification WO 88/03015, aqueous preparations are described, which contain ubidecarenone (ubiquinone-10) and specific amphipathic compounds, which form micellar and liposominal aggregates with the ubidecarenone. When applied to the skin, various cosmetic effects are reported to be obtained, including stimulation of hair growth.

From EP-A-0 100 915 a hair growth agent is known, which contains ubiquinone (coenzyme Qₙ n=7-10) and optionally also additionally vasodilators peripherally effective in the skin, such as carpronium chloride, Vitamin E nicotinate and benzyl nicotinate.

From the literature, additional hair-growth antihypertensives are additionally known, which belong to the group of potassium channel openers. One such, very well-known active substance is minoxidil. Potassium channel openers, besides other substance groups, belong to the vasodilators.

From U.S. Pat. No. 4,139,619 and EP-A-0 188 793, hair growth agents with minoxidil as active substance are known, which are applied topically.

From the literature (Merck Index, 12th edition, Abstract no 4125), the 5-alpha reductase inhibitor finasteride (INN) is also known, which in animal experimentation is reported to be effective with regard to hair growth.

However none of these documents could have prepared the way to the present invention.

Dandruff is regarded as a further unpleasant cosmetic disorder of the scalp. Many proposals have also already been made for its treatment.

Therefore topical cosmetic preparations are particularly desirable, which not only have a favourable effect on the hair growth, but also care for the scalp and reduce or prevent seborrhoeic symptoms, in particular dandruff.

Known hair treatment agents often have disadvantages. Their effect is frequently not satisfactory or they represent a health risk, particularly in the case of constant application.

The task of the invention is therefore to make available better agents for influencing hair growth and for the prophylaxis and treatment of seborrhoeic symptoms, in particular dandruff.

These tasks are solved according to the invention.

An object of the invention is the use of one or more compounds from the group of bioquinones

a) in combination with one or more compounds from the group of potassium channel openers and/or

b) in combination with one or more compounds from the group of 5-alpha reductase inhibitors, for the production of cosmetic or dermatological preparations for treating the scalp and the hair, in order to prolong the anagen phase and/or to treat and prevent seborrhoeic symptoms, optionally with the additional use of one or more compounds from the group formed by carnitine, arginine, succinic acid, folic acid, conjugated fatty acids and their respective derivatives, in addition to antioxidants.

A further object of the invention is preparations with a content of one or more compounds from the group of bioquinones.

a) in combination with one or more compounds from the group of potassium channel openers and/or

b) in combination with one or more compounds from the group of 5-alpha reductase inhibitors, optionally with a content of one or more compounds from the group formed by carnitine, arginine, succinic acid, folic acid, conjugated fatty acids and their respective derivatives, in addition to antioxidants.

A further object of the invention is the use of one or more compounds from the group of bioquinones

a) in combination with one or more compounds from the group of potassium channel openers and/or

b) in combination with one or more compounds from the group of 5-alpha reductase inhibitors, in order to prolong the anagen phase and/or to treat and prevent seborrhoeic symptoms, optionally with the additional use of one or more compounds from the group formed by carnitine, arginine, succinic acid, folic acid, conjugated fatty acids and their respective derivatives, in addition to antioxidants.

A further object of the invention is combinations of one or more compounds from the group of bioquinones

a) in combination with one or more compounds from the group of potassium channel openers and/or

b) in combination with one or more compounds from the group of 5-alpha reductase inhibitors, optionally combined with one or more compounds from the group formed by carnitine, arginine, succinic acid, folic acid, conjugated fatty acids and their respective derivatives, in addition to antioxidants.

Combinations or uses for which the following active substances are combined and/or used are preferred:

a) Ubiquinones, in particular coenzyme Q-10, and minoxidil, or

b) Ubiquinones, in particular coenzyme Q-10, and finasteride and/or gamma linoleic acid, or

c) Ubiquinones, in particular coenzyme Q-10, and minoxidil and finasteride and/or gamma linoleic acid,

optionally with the additional use of one or more compounds from the group formed by carnitine, arginine, succinic acid, folic acid, conjugated fatty acids and their respective derivatives, in addition to antioxidants, but preferably with additional use of carnitine and/or conjugated fatty acids, in particular conjugated linoleic acid, or their respective derivatives.

The following active-substance combinations can for example be present (with at least one compound from the desired active substance group):

1) Bioquinone and potassium channel opener,

2) Bioquinone and 5-alpha reductase inhibitor,

3) Bioquinone and potassium channel opener and 5-alpha reductase inhibitor.

The agents according to the invention are preferably applied topically.

The term “bioquinones” refers to differently substituted prenylated quinones which occur in humans, animals and plants.

Preferred bioquinones are ubiquinones, plastoquinones and boviquinones, but ubiquinones in particular.

Suitable ubiquinones are characterised by the structural formula

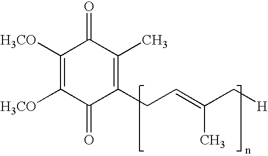

(n=1-10) and represent the bioquinones which are most widely distributed and hence best examined. Ubiquinones are designated, according to the number of isoprene units connected to the side chain, as Q-1, Q-2, Q-3 etc. or according to the number of C-atoms as U-5, U-10, U-15 etc. They occur preferably with specific chain lengths, e.g. in some micro organisms and yeasts, with n=6. In most mammals including humans, Q-10 predominates.

Coenzyme Q-10 is preferred. This is characterised by the following structural formula:

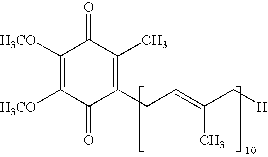

Ubiquinones serve the organisms as electron transferors in the respiratory chain. They are found in the mitochondria where they enable the cyclic oxidation and reduction of the substrates of the citric acid cycle.

Suitable plastoquinones have the general structural formula (n=1-10).

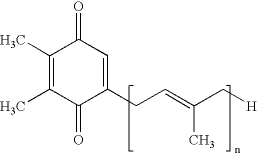

They can be isolated from chloroplasts and play a role as redox substrates in photosynthesis in the case of cyclic and non-cyclic electron transport, wherein they change reversibly into the corresponding hydroquinones (plastoquinol). Plastoquinones differ in the number n of isoprene radicals and are designated correspondingly, e.g. PQ-9 (n=9). Other plastoquinones also exist, with different substituents on the quinone ring.

According to the invention, the preferred bioquinone is coenzyme Q-10.

It is advantageous, in the finished preparations, to choose concentrations of 0.000001-10 wt. %, in particular 0.001-1 wt. % of one or more bioquinones, preferably coenzyme Q-10, in each case relative to the total weight of the preparations.

A number of potassium channel openers are described in the literature.

According to the invention, the following substances are suitable:

Minoxidil

Pinacidil

Diazoxide

Cromakalim

Rilmakalim

Nicorandil

Flupirtine

KRN2391

P-1075

ZD6169

RP-49,356

YM934

MCC-134 and

SKP 450.

The following active substances are especially preferred:

Minoxidil, e.g. minoxidil sulphate

Pinacidil

Diazoxide

Cromakalim

Rilmakalim

Nicorandil

Flupirtine,

but minoxidil and/or pinacidil in particular.

Potassium channel openers are preferably contained in the cosmetic and dermatological preparations according to the invention, in quantities of 0.01 to 20 wt. %, especially preferably 0.01 to 5 wt. %, in particular 1 to 3 wt. %, in each case relative to the total preparation.

The relation of the quantities by weight of the combination active substances bioquinones/potassium channel openers to each other can vary widely in the preparations. For example, it can amount to 1/10 to 10/1, or 5/1 to 1/5. However it can preferably amount to 1/2 to 2/1 and in particular 1/1.

5-alpha-reductase inhibitors are described in the literature. They inhibit an enzyme which brings about the conversion of testosterone into the more potent androgen 5-alpha-dihydrotestosterone.

Suitable 5-alpha-reductase inhibitors are for example steroidal but also non-steroidal 5-alpha-reductase inhibitors as described in the literature (W. Chen. et al, Dermatologie, 1996, 193: 177-184).

Steroidal 5-alpha-reductase inhibitors are, for example, finasteride, turosteride, MK-434, MK-963, epristeride and MK-386.

Non-steroidal 5-alpha-reductase inhibitors are, for example, ONO-3805, LY191704, FK 143, polyunsaturated fatty acids, zinc ions, e.g. the water-soluble salts of inorganic acids, catechin, epicatechin, extracts of tea, e.g. of green or black tea, epicatechin-3-gallate or epigallocatechin-3-gallate.

Suitable polyunsaturated fatty acids can, for example, in each case possess up to 24, preferably up to 18, in particular up to 12 carbon atoms, and e.g. straight-chained or branched alkyl-monocarboxylic acid or cycloalkyl-monocarboxylic acid. They can, for example, possess two to six multiple bonds, in particular double bonds.

Especially preferable are gamma linoleic acid (GLA), zinc salts, e.g. zinc chloride, tea and green tea extracts, catechins, e.g. epicatechin-3-gallate and/or epigallocatechin-3-gallate.

Finasteride (17β-(N-tert-butylcarbamoyl)-4-aza-5α-androstan-1-en-3-one) is preferred, as already cited above.

5-alpha-reductase inhibitors are preferably contained in the cosmetic and dermatological preparations according to the invention preferably in quantities of 0.01 to 10 wt. %, especially preferably 0.1 to 1 wt. %, in particular 0.4 to 0.6 wt. % in each case relative to the total preparation.

The ratio of the quantities by weight of the combination active substances bioquinones/5-alpha-reductase inhibitors can vary considerably in the preparations. For example, it can amount to 1/10 to 10/1, or 5/1 to 1/5. However it can also amount to 1/2 to 2/1 and in particular 1/1.

Preparations with the following active substance combinations are preferred:

1) Q-10 and minoxidil

2) Q-10 and finasteride and/or gamma linoleic acid

3) Q-10 and minoxidil and finasteride and/or gamma linoleic acid

The above mentioned weight ratios are also preferably used for these.

To the preparations, which contain at least one bioquinone and in combination at least one potassium channel opener and/or a 5-alpha-reductase inhibitor as active substances, can preferably be added further active substances such as carnitine, arginine, succinic acid, conjugated fatty acid and/or folic acid, and/or also their derivatives, and/or one or more compounds from the antioxidants group, e.g. to improve the effect.

Suitable derivatives of carnitine are, for example, O-acylcarnitine with straight-chained or branched C₁-C₁₂ alkyl groups of the alkylcarbonyl residue (acyl residue). Acetyl carnitine and its derivatives, e.g. as indicated below, are preferred. Carnitine and the acyl carnitines can also be used as salts, acid addition salts, esters or amides.

Preferred salts are water-soluble salts, e.g. sodium, potassium and ammonium salts. This also applies to the acid addition salts. Suitable acid addition salts are obtained with inorganic and organic acids. The hydrochlorides, sulphates, acetates, caprylates or citrates are preferred.

Suitable esters are, e.g. such as are obtained with short-chained, medium-chained or long-chained alcohols, preferably mono-alcohols, but in particular methanol, ethanol or propanol. The ethyl esters are preferred.

Preferred amides are short- or medium-chained or long-chained mono- and di-alkylamides.

Alkyls of the above substituents contain, e.g. up to 20, preferably up to 6 carbon atoms, in particular one or two carbon atoms.

Carnitine and/or its derivatives are contained in the preparations according to the invention, preferably in quantities of 0.00001 to 10 wt. %, in particular 0.01 to 1.5 wt. %, in each case relative to the total weight of the preparations.

Arginine can be present as racemate or in optically active form (D- or L-). L-arginine and/or its derivatives are preferred.

Suitable derivatives of the arginine are e.g. its salts, acid addition salts, esters or amides.

Preferred salts of arginine are water-soluble salts, e.g. sodium, potassium and ammonium salts. This also applies to acid addition salts. Suitable acid addition salts are obtained with inorganic or organic acids. The hydrochlorides, sulphates, acetates, caprylates or citrates are preferred.

Suitable arginine esters are e.g. those which are obtained with short-chained or medium-chained or long-chained alcohols, preferably mono-alcohols, but in particular methanol, ethanol or propanol. The ethyl esters are preferred.

Preferred amides are short- or medium-chained or long-chained mono- and di-alkylamides.

Alkyls of the above substituents contain e.g. up to 20, preferably up to 6 carbon atoms, in particular one or two carbon atoms.

Arginine and its derivatives are also characterised by a particularly good skin penetration capacity.

Arginine and its derivatives are contained in the cosmetic and dermatological preparations according to the invention, preferably in quantities of 0.01 to 30 wt. %, especially preferably 0.01 to 10 wt. %, in particular 0.1-7.5 wt. %, in each case relative to the total preparation.

Suitable derivatives of succinic acid are, for example, the succinates, i.e. the succinic acid esters and salts, as well as the respective hydrogen succinates, and also the acid addition salts, but also succinic acid amides or the corresponding hydrogen amides.

Preferred salts, acid addition salts or esters are such as have already been described for the arginine derivatives.

Disodium succinate is preferred.

Succinic acid and/or its derivatives are contained in the cosmetic and dermatological preparations according to the invention, preferably in quantities of 0.001 to 30 wt. %, especially preferably 0.01 to 20 wt. %, especially preferably 0.01 to 10 wt. %, in particular 0.1-7.5 wt. %, in each case relative to the total preparation.

Suitable derivatives of folic acid are for example its salts, acid addition salts, esters or amides. Such salts, acid addition salts, esters or amides as have already been described for the arginine derivatives are preferred.

Folic acid is preferably used.

Folic acid and/or its derivatives are contained in the preparations according to the invention, preferably in quantities of 0.0001 to 5 wt. %, in particular 0.01 to 1.5 wt. %, in each case relative to the total weight of the preparations.

Further important components, which in addition to carnitine, arginine, succinic acid and folic acid, are suitable e.g. for improving the energy metabolism of the hair roots, are conjugated fatty acids, i.e. monocarboxylic acids with at least two conjugated multiple bonds, in particular double bonds and their derivatives. These are also referred to as “CFAs” here. All geometric isomer forms and position isomer forms as well as the mixtures of such compounds and their derivatives, for example the salts, esters or amides are suitable.

Such conjugated fatty acids are known and can be obtained according to known methods, for example by alkaline isomerisation of the corresponding fatty acids with isolated multiple bonds/double bonds.

Suitable fatty acids can for example possess up to 24, preferably up to 18, in particular up to 12 carbon atoms and can be e.g. straight-chained or branched alkyl monocarboxylic acids or cycloalkyl-monocarboxylic acids. These can for example possess 2 to 6 conjugated multiple bonds, in particular double bonds.

Preferred salts are water-soluble salts, e.g. sodium, potassium and ammonium salts.

Suitable esters are e.g. such, as are obtained with short-chained, medium-chained or long-chained alcohols, preferably mono-alcohols, but in particular methanol, ethanol or propanol. The ethyl esters are preferred.

Preferred amides are short- or medium-chained or long-chained mono- and di-alkylamides.

Alkyls of the above substituents receive e.g. up to 20, preferably up to 6 carbon atoms, in particular one or two carbon atoms.

A preferred CFA which e.g. improves the energy metabolism of the hair roots, in addition to carnitine, arginine, succinic acid and folic acid, is conjugated linoleic acid, also known as “CLA”, in all its geometric isomer forms and position isomer forms as well as the mixtures of such compounds and their derivatives, in particular as described above.

Linoleic acid (cis, cis-9,12-octadecadienoic acid) has no conjugated double bonds. Thistle oil and sunflower oil possess a high proportion of this acid. For example, from the linoleic acid of these raw materials, the conjugated compounds are obtained in a known manner by alkaline isomerisation. A preferred isomer mixture is also described in the literature (Lipids, vol. 34, No. 9 (1999) p. 997-1000, Table 1). The conjugated double bonds of the CFAs preferably lie within the range of carbon atoms 9 to 12.

CFAs or CLA and/or their derivatives are contained in the preparations according to the invention, preferably in quantities of 0.0001 to 5 wt. %, in particular 0.01 to 1.5 wt. %, in each case relative to the total weight of the preparations.

Carnitine and/or its derivatives in combination with CFAs and/or their derivatives are preferably used, in particular in the respective quantities by weight indicated. The ratio of the quantities by weight of these combination active substances carnitine/CFAs can vary widely in the preparations. For example it can amount to 1/10 to 10/1, or 5/1 to 1/5. However it can also preferably amount to 1/2 to 2/1 and in particular 1/1.

One consequence of extending the time for the anagen phase of the hair growth cycle is an increase in the density of the hairs, i.e. on a given area unit of the scalp, there is a larger number of hairs, and of intact hairs. In addition the length of the hairs increases, because there is more time available for further growth.

The prolonging of the anagen phase according to the invention is achieved in the case of normal hair growth, but also in the case of a disturbed, shortened anagen phase, i.e. also in conditions which are accompanied by low hair density.

This also achieves a higher percentage of the hairs present being in the anagen phase.

The formation of dandruff is also prevented or considerably reduced according to the invention.

For use, the preparations according to the invention are preferably applied directly to the scalp, in the manner known for such agents, for example twice daily.

The following are suitable e.g.: solutions, gels, ointments, suspensions or emulsions such as cremes or lotions with a content of the active substances according to the invention.

Hair treatment agents with a content of the active substances according to the invention are also suitable, in particular those which remain in the hair or are used to take effect over a fairly long period. Also, in this way, the active substances get into or onto the scalp or into the region of the hair roots. Hair treatment agents which come into contact with the skin or hair for only a short period, e.g. shampoos, can for example contain higher percentages of active substances.

Hair treatment agents are for example shampoos, hair-care products such as hair lotions, hairstyling products, conditioners, hair treatments, treatment packages, hair setting lotions, such as mousses, hair spray, hair lacquer, perms and dyes.

Cosmetic and, if appropriate, dermatological preparations according to the invention can exist in various forms. They can e.g. be in the form of a solution, an anhydrous preparation, an emulsion or microemulsion of the water-in-oil (W/O) type, or of the oil-in-water (O/W) type, a multiple emulsion, for example of the water-in-oil-in-water (W/O/W/) type, a gel, a solid stick, an ointment or an aerosol. It is also advantageous, according to the invention, to administer one or more bioquinones in encapsulated form, e.g. in collagen matrices and other standard encapsulation materials, e.g. as cellulose encapsulations, in gelatine, wax matrices or liposomally encapsulated. In particular wax matrices, as described in DE-OS 43 08 282 have proved particularly favourable.

The cosmetic and dermatological preparations according to the invention can contain cosmetic adjuvant substances, as normally used in such preparations, e.g. preservatives, bactericides, perfumes, substances to prevent foaming, dyes, pigments which have a colouring effect, thickening agents, surfactants, emulsifiers, softening, moistening and/or moisture-containing substances, fats, oils, waxes or other normal components of a cosmetic or dermatological formulation such as alcohols, polyols, polymers, foam stabilisers, electrolytes, organic solvents or silicone derivatives.

In particular, bioquinones used according to the invention can also be combined with antioxidants, including radical scavengers.

Such antioxidants are advantageously selected from the group consisting of amino acids (e.g. glycine, histidine, tyrosine, tryptophane) and their derivatives, imidazoles (e.g. urocanic acid) and their derivatives, peptides such as D,L-carnosine, D-carnosine, L-carnosine and their derivatives (e.g. Anserin), carotinoids, carotenes (e.g. α-carotene, β-carotene, lycopene) and their derivatives, chlorogenic acid and its derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl-, N-acetyl-, methyl-, ethyl-, propyl-, amyl-, butyl- and lauryl-, palmitoyl-, oleyl-, γ-linoleyl-, cholesteryl- and glyceryl esters) and their salts, dilaurylthiodipropionate, distearylthiodipropionate, thiodipropionic acid and their derivatives (esters, ethers, peptides, lipids, nucleotides, nucleosides and salts) as well as sulphoximine compounds (e.g. buthionine sulphoximines, homocysteine sulphoximine, buthionine sulphone, penta-, hexa, heptathionine sulphoximine) in very low, well tolerated doses (e.g. pmol to μmol/kg), furthermore (metal)-chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. linoleic acid, oleic acid), tocopherols and derivatives (e.g. vitamin E acetate), vitamin A and derivatives (vitamin A palmitate) and coniferyl benzoate of benzoic resin, flavonoids, e.g. alpha-glycosyl rutin, rutinic acid and their derivatives, butylhydroxytoluene, butylhydroxyanisol, nordihydroguajak resin acid, nordihydroguajeret acid, trihydroxybutyrophenone, uric acid and their derivatives, mannoses, and their derivatives, sesamol, sesamolin, zinc and its derivatives (e.g. ZnO, ZnSO₄), selenium and its derivatives (e.g. selenium methionine) stilbenes and their derivatives (e.g. stilbene oxide, trans-stilbene oxide) and the suitable derivatives according to the invention (salts, esters, ethers, sugars, nucleotides, nucleosides, peptides and lipids) of these named active substances.

The quantity of the abovementioned antioxidants (one or more compounds) in the preparations preferably amounts to 0.001 to 30 wt. %, especially preferably 0.05-20 wt. %, and in particular 1-10 wt. % relative to the total weight of the preparation.

If vitamin E and/or its derivatives represent the additional antioxidant or antioxidants, it is advantageous to choose their respective concentrations from the range 0.001-10 wt. %, relative to the total weight of the formulation.

If vitamin A and/or vitamin A derivatives, and/or carotenes and/or their derivatives represent the additional antioxidant or antioxidants, it is advantageous to choose their respective concentrations from the range 0.001-10 wt. %, relative to the total weight of the formulation.

According to the invention, emulsions are an advantageous embodiment of the invention and contain e.g. the named fats, oils, waxes and other lipoids, as well as water and an emulsifier, as normally used for such a type of formulation.

The lipid phase can be advantageously chosen from the following substance group:

mineral oils, mineral waxes

oils, such as triglycerides of caprinic or caprylic acid, as well as natural oils such as e.g. ricinus oil;

fats, waxes and other natural and synthetic lipoids, preferably esters of fatty acids with alcohols of low C-number, e.g. with isopropanol, propylene glycol or glycerine, or esters of fatty alcohols with alkanoic acids of low C-number or with fatty acids;

alkylbenzoates;

silicone oils such as dimethylpolysiloxane, diethylpolysiloxane, diphenylpolysiloxane and mixed forms thereof.

The oil phase of the emulsions, oleogels/hydrodispersions or lipodispersions in accordance with the present invention is advantageously chosen from the group of esters of saturated and/or unsaturated, branched and/or unbranched alkane carboxylic acids of a chain length of 3 to 30 C-atoms and saturated and/or unsaturated, branched and/or unbranched alcohols of a chain length of 3 to 30 C-atoms, from the group of esters of aromatic carboxylic acids and saturated and/or unsaturated, branched and/or unbranched alcohols of a chain length of 3 to 30 C-atoms. Such ester oils can then be advantageously chosen from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethyl hexyl palmitate, 2-ethyl hexyl laurate, 2-hexyl decyl stearate, 2-octyl dodecyl palmitate, oleyl oleate, oleyl erucate, erucyl oleate, erucyl erucate as well as synthetic, semi-synthetic and natural mixtures of such esters, e.g. jojoba oil.

Furthermore, the oil phase can be advantageously selected from the group of branched and unbranched hydrocarbons and waxes, the silicone oils, the dialkyl ethers, the group of saturated or unsaturated, branched or unbranched alcohols, as well as the fatty acid triglycerides, in particular the triglycerine esters of saturated and/or unsaturated, branched and/or unbranched alkane carboxylic acids of a chain length of 8 to 24, in particular 12-18 C-atoms. The fatty acid triglycerides can for example be advantageously chosen from the group of synthetic, semi-synthetic and natural oils, e.g. olive oil, sunflower oil, soya bean oil, groundnut oil, rape seed oil, almond oil, palm oil, coconut oil, palm nut oil and suchlike.

Also, any mixtures of such oil and wax components can be advantageously used in accordance with the present invention. It may possibly also be advantageous to use waxes, for example cetyl palmitate as sole lipid component of the oil phase.

The oil phase is advantageously selected from the group 2-ethylhexyl isostearate, octyl dodecanol, isotridecyl isononanoate, isoeicosane, 2-ethylhexyl cocoate, C₁₂₋₁₅-alkyl benzoate, capryl-caprinic acid triglyceride, dicaprylyl ether.

Mixtures of C₁₂₋₁₅-alkyl benzoate, and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous.

Of the hydrocarbons, paraffin oil, squalane and squalene can be advantageously used in accordance with the invention.

The oil phase can, furthermore, advantageously have a content of cyclic or linear silicone oils or consist completely of such oils, it being preferable however, apart from the silicone oil or the silicone oils, to use an additional content of other oil phase components.

Cyclomethicone (octamethylcyclotetrasiloxane) is advantageously utilised as the silicone oil to be used according to the invention. But other silicone oils can also be advantageously used in accordance with the invention, for example, hexamethyl cyclotrisiloxane, polydimethyl siloxane, poly(methylphenyl siloxane).

Also particularly advantageous are mixtures of cyclomethicone and isotridecyl isononanoate, and of cyclomethicone and 2-ethylhexyl isostearate.

The aqueous preparations according to the invention and/or the aqueous phase of the preparations according to the invention optionally advantageously contain alcohols, diols or polyols of low C-number, as well as their ethers, preferably ethanol, isopropanol, propylene glycol, glycerine, ethylene glycol, ethylene glycol monoethyl- or -monobutyl ethers, propylene glycol monomethyl, -monoethyl or -monobutyl ethers, diethylene glycol monomethyl- or -monoethyl ethers and analogous products, also alcohols of low C-number, e.g. ethanol, isopropanol, 1,2-propane diol, glycerine and in particular one or more thickening agents, which can advantageously be chosen from the group: silicium dioxide, aluminium silicates, polysaccharides and/or their derivatives, e.g. hyaluronic acid, xanthan gum, hydroxypropylmethyl cellulose, especially advantageously from the group of the polyacrylates, preferably a polyacrylate from the group of so-called carbopols, for example carbopols of types 980, 981, 1382, 2984, 5984, in each case individually or in combination.

In particular, mixtures of the above-mentioned solvents are used. In the case of alcoholic solvents, water can be an additional component.

In the technical sense, the term “gels” is understood to mean: disperse systems which are relatively stable in shape and easily deformable, made from at least two components, which as a rule consist of one—usually solid—colloidally distributed substance of long-chained molecule groups (e.g. gelatines, silicic acid, polysaccharides) forming a framework, and one liquid dispersion agent (e.g. water). The colloidally distributed substance is often described as a thickening or gelling agent. It forms a physical network in the dispersion agent, wherein individual particles existing colloidally can be more or less firmly connected to each other via electrostatic interaction. The dispersion agent, which surrounds the network, is characterised by electrostatic affinity to the gelling agent, i.e. a predominantly polar (in particular hydrophilic) gelling agent gels preferably a polar dispersion agent (in particular: water), whereas a predominantly non-polar gelling agent gels preferably non-polar dispersion agents.

Strong electrostatic interactions, which are for example realised in hydrogen-bridge bonds between gelling agents and dispersion agents, but also between dispersion agent molecules amongst themselves, can also lead to strong cross-linking of the dispersion agent.

Hydrogels can consist of up to almost 100% water (in addition for example to approx. 0.2-1.0% of a gelling agent) and still possess a quite solid consistency. The water content is present in ice-like structural elements, which is completely in keeping with the etymology of the word “gel” [from Latin “gelatum”=“frozen”, via the expression “gelatina” used in alchemy (16th century) to the modern “gelatine”].

Suitable propellants for preparations according to the invention, which can be sprayed from aerosol containers, include the conventionally known, highly volatile, liquefied propellants, for example hydrocarbons (propane, butane, isobutane) which can be used alone or in mixture with each other. Compressed air can also be used advantageously.

Preparations according to the invention can in addition advantageously contain substances which absorb UV radiation in the UVB range, the total quantity of the filtering substances amounting to e.g. 0.1 wt. % to 30 wt. %, preferably 0.5 to 10 wt. %, and in particular 1.0 to 6.0 wt. %, relative to the total weight of the preparations, to provide cosmetic preparations which protect the hair and/or skin from the whole range of ultraviolet radiation. They can also be used as sun-protection products for the hair or skin.

If the preparations according to the invention contain UVB-filtering substances, these can be oil-soluble or water-soluble. Advantageous oil-soluble UVB filters according to the invention are, e.g.:

3-benzylidene camphor derivatives, preferably 3-(4-methylbenzylidene) camphor, 3-benzylidene camphor;

4-aminobenzoic acid derivatives, preferably 4-(dimethylamino)-benzoic acid(2-ethylhexyl)ester, 4-(dimethylamino)benzoic acid amyl ester;

esters of cinnamic acid, preferably 4-methoxy cinnamic acid(2-ethylhexyl)ester, 4-methoxy cinnamic acid isopentyl ester;

esters of salicylic acid, preferably salicylic acid(2-ethylhexyl)ester, salicylic acid(4-isopropylbenzyl)ester, salicylic acid homomenthyl ester,

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably 4-methoxybenzalmalonic acid di(2-ethylhexyl)ester, -2,4,6-trianilino-(-p-carbo-2′-ethyl-1′-hexyloxy)-1,3,5 triazine.

Advantageous water-soluble UVB filters include e.g.:

Salts of 2-phenylbenzimidazol-5-sulphonic acid such as its sodium, potassium, or its triethanol ammonium salt, and the sulphonic acid itself;

Sulphonic acid derivatives of benzophenone, preferably 2-hydroxy-4-methoxybenzophenone-5-sulphonic acid and its salts;

Sulphonic acid derivatives of 3-benzylidene camphor, such as e.g. 4-(2-oxo-3-bornylidene methyl)benzene sulphonic acid, 2-methyl-5-(2-oxo-3-bornylidene methyl)sulphonic acid and its salts, and 1,4-di(2-oxo-10-sulpho-3-bornylidene methyl)benzene and its salts (the corresponding 10-sulphato compounds, for example the corresponding sodium, potassium or triethanol ammonium salt), also described as benzene-1,4-di(2-oxo-3-bornylidene methyl-10-sulphonic acid.

The list of named UVB filters, which can be used in combination with the active substance combinations according to the invention, is not, of course, intended to be limiting.

A subject of the invention is also the use of a combination of the bioquinones used according to the invention with at least one UVB filter as antioxidant, and/or the use of a combination of the bioquinones used according to the invention with at least one UVB filter as antioxidant in a cosmetic or dermatological preparation for use on hair.

It can also be advantageous, to combine the bioquinones used according to the invention with UVA filters, which have hitherto normally been contained in cosmetic preparations. These substances are preferably dibenzoyl methane derivatives, in particular 1-(4′-tert.butylphenyl-3-(4′-methoxyphenyl)propane-1,3-dione and 1-phenyl-3-(4′-isopropylphenyl-propane-1,3-dione. These combinations and/or preparations which contain these combinations are also a subject of the invention. The quantities utilised for the UVB combination can be used.

The cosmetic and dermatological preparations according to the invention can contain cosmetic adjuvant substances, as normally used in such preparations, e.g. preservatives, bactericides, perfumes, substances to prevent foaming, dyes, pigments which have a colouring effect, thickening agents, surfactants, emulsifiers, softening, moistening and/or moisture-containing substances, fats, oils, waxes or other normal components of a cosmetic or dermatological formulation such as alcohols, polyols, polymers, foam stabilisers, electrolytes, organic solvents, silicone derivatives and/or comb polymers.

In cosmetic preparations used to set the hair, such as e.g. hair sprays, hair lacquer, setting mousses, setting lotions, styling gels etc., the comb polymers to be used according to the invention can preferably be used in concentrations of 0.5 to 30 percent by weight.

The compounds according to the invention for setting the hair can be in the form of hairsprays or mousse aerosols, and contain the additives which are normal for these and which correspond to the state of the art, providing there is corresponding compatibility. These are for example further solvents such as low-density polyalcohols and their toxicologically well tolerated ethers and esters, softeners, silicones of high and low volatility, branched/unbranched hydrocarbons of high and low volatility, emulsifiers, antioxidants, waxes, stabilisers, pH-value regulators, dyes, agents to give consistency, antistatics, UV absorbers, perfumes, etc.

If the compound according to the invention is to be used as hairspray or mousse aerosol, a propellant is usually added. The usual propellants are the lower alkanes, for example, propane, butane or isobutane, dimethyl ether, nitrogen, nitrogen dioxide or carbon dioxide or mixtures of these substances.

If used in mechanical spray or foaming devices, for example spray pumps or manual foaming pumps/squeeze systems, there is normally no need for the propellant.

The cosmetic and dermatological preparations according to the invention can for example also be shampoos, blow-drying or hair-setting preparations, colouring preparations, and styling or treatment lotions.

Preparations according to the invention can, if appropriate, be advantageously characterised by a surfactants content. Surfactants are amphiphilic substances which can dissolve organic, non-polar substances in water. Due to their specific molecular structure, with at least one hydrophilic and one hydrophobic molecule part, they reduce the surface tension of the water, ensuring moistening of the skin, facilitating the removal and dissolving of dirt, making rinsing easy, and—if desired—regulating foam.

The hydrophilic part of a surfactant molecule usually comprises polar functional groups, for example —COO⁻, OSO₃²⁻, —SO₃⁻, whilst the hydrophobic parts are generally constituted by non-polar hydrocarbon radicals. Surfactants are generally classified according to the nature and charge of the hydrophilic part of the molecule, it being possible to differentiate between four groups:

anionic surfactants,

cationic surfactants,

amphoteric surfactants and

non-ionic surfactants.

Anionic surfactants generally have carboxylate, sulphate or sulphonate groups as functional groups. In aqueous solution they form, in an acidic or neutral medium, negatively charged organic ions. Cationic surfactants are almost exclusively characterised by the presence of a quaternary ammonium group. In aqueous solution they form, in an acidic or neutral medium, positively charged organic ions. Amphoteric surfactants contain both anionic and cationic groups and therefore, in an aqueous solution, behave as anionic or cationic surfactants depending on the pH value. In a strongly acidic medium they possess a positive, and in an alkaline medium a negative charge. In the neutral pH range, on the other hand, they are zwitterionic, as made clear by the following example:

| | | |
|---|---|---|
| RNH₂⁺CH₂CH₂COOH X⁻ | (at pH = 2) | X⁻ = any anion, e.g. Cl⁻ |
| RNH₂⁺CH₂CH₂COO⁻ | (at pH = 7) | |
| RNHCH₂CH₂COO⁻ B⁺ | (at pH = 12) | B⁺ = any cation, e.g. Na⁺ |

Polyether chains are typical of non-ionic surfactants. Non-ionic surfactants form no ions in an aqueous medium.

A. Anionic Surfactants

Anionic surfactants to be used advantageously are

acyl amino acids (and their salts), such as:

1. Acyl glutamates, for example sodium acyl glutamate, Di-TEA-palmitoyl aspartate and sodium caprylic/capric glutamate,

2. Acyl peptides, for example palmitoyl-hydrolysed milk protein, sodium cocoyl-hydrolysed soy protein and sodium/potassium cocoyl-hydrolysed collagen,

3. Sarcosinates, for example myristoyl sarcosine, TEA-lauroyl sarcosinate, sodium lauroyl sarcosinate and sodium cocoyl sarcosinate.

4. Taurates, for example sodium lauroyl taurate and sodium methyl cocoyl laurate.

5. Acyl lactylates, lauroyl lactylate, caproyl lactylate

6. Alaninates

Carboxylic acids and derivatives, such as

1. Carboxylic acids, for example lauric acid, aluminium stearate, magnesium alkanolate and zinc undecylenate,

2. Ester carboxylic acids, for example calcium stearoyl lactylate, laureth-6 citrate and sodium PEG-4 lauramide carboxylate,

3. Ether carboxylic acids, for example sodium laureth-13 carboxylate and sodium PEG-6 cocamide carboxylate.

Phosphoric acid esters and salts, for example DEA-oleth-10-phosphate and dilaureth-4 phosphate,

Sulphonic acids and salts, such as

1. Acyl-isethionate, e.g. sodium/ammonium cocoyl isethionate,

2. Alkylaryl sulphonates,

3. Alkyl sulphonates, for example sodium coco-monoglyceride sulphate, sodium C₁₂₋₁₄, olefin sulphonate, sodium lauryl sulphoacetate and magnesium PEG-3 cocamide sulphate,

4. Sulphosuccinates, for example dioctyl sodium sulphosuccinate, disodium laureth sulphosuccinate, disodium lauryl sulphosuccinate and disodium undecylene amido MEA-sulphosuccinate,

and

Sulphuric acid esters, such as

1. Alkyl ether sulphate, for example sodium-, ammonium-, magnesium-, MIPA-, TIPA-, laureth sulphate, sodium myreth sulphate and sodium C₁₂₋₁₃ pareth sulphate.

2. Alkyl sulphates, for example sodium-, ammonium- and TEA- lauryl sulphate.

B. Cationic Surfactants

Cationic surfactants that may possibly be used advantageously include

1. Alkyl amines,

2. Alkylimidazoles,

3. Ethoxylated amines and

4. Quaternery surfactants

5. Esterquats

Quaternary surfactants contain at least one N-atom, which is covalently bonded with 4 alkyl- or aryl groups. This leads, independently of the pH value, to a positive charge. The following are advantageous: alkyl betaine, alkylamidopropyl betaine and alkyl-amidopropyl hydoxysulphane. The cationic surfactants used according to the invention can further be advantageously selected from the group of quaternary ammonium compounds, in particular benzyltrialkyl ammonium chloride or -bromide, such as for example benzyldimethylstearyl ammonium chloride, also alkyltrialkyl ammonium salts, for example cetyltrimethyl ammonium chloride or -bromide, alkyldimethyl-hydroxyethyl ammonium chloride or -bromide, dialkyldimethyl ammonium chloride or -bromide, alkyl amidethyl trimethyl ammonium ether sulphate, alkyl pyridium salts, for example lauryl- or cetyl pyrimidinium chloride, imidazolin derivatives and compounds with cationic character such as aminoxide, for example alkyl dimethyl aminoxide or alkylaminoethyl dimethyl aminoxide. Cetyl trimethyl ammonium salts can be used particularly advantageously.

C. Amphoteric Surfactants

Amphoteric surfactants to be used advantageously include

1. Acyl-/dialkyl ethylene diamine, for example sodium acyl amphoacetate, disodium acyl amphodipropionate, disodium alkyl amphodiacetate, sodium acyl amphohydroxypropyl sulphonate, disodium acyl amphodiacetate and sodium acyl amphopropionate,

2. N-alkyl amino acids, for example aminopropyl alkyl glutamide, alkyl amino propionic acid, sodium alkyl imidodipropionate and lauroamphocarboxyglycinate.

D. Non-Ionic Surfactants

Non-ionic surfactants to be used advantageously include

1. Alcohols

2. Alkanomides, such as cocamides MEA/DEA/MIPA,

3. Aminoxides, such as cocoamidopropyl aminoxide,

4. Esters, which are produced by the esterification of carboxylic acids with ethylene oxide, glycerine, sorbitan, or other alcohols.

5. Ethers, for example ethoxylated/propoxylated alcohols, ethoxylated/propoxylated esters, ethoxylated/propoxylated glycerine esters, ethoxylated/propoxylated cholesterins, ethoxylated/propoxylated triglyceride esters, ethoxylated/propoxylated lanolin, ethoxylated/propoxylated polysiloxanes, propoxylated POE ethers and alkyl polyglycosides such as lauryl glycoside, decyl glycoside and cocoglycoside.

6. Sucrose esters, -ethers

7. Polyglycerine esters, diglycerine esters, monoglycerine esters

8. Methyl glucose esters, esters of hydroxy acids.

Also advantageous is the use of a combination of anionic and/or amphoteric surfactants with one or more non-ionic surfactants.

In general, within the meaning of the present invention, the use of anionic, amphoteric and/or non-ionic surfactants is preferred over the use of cationic surfactants.

The cosmetic and dermatological [preparations] contain active substances and auxiliary substances, such as those normally used for this type of hair care and hair treatment preparations. As auxiliary substances, preservatives, surfactant substances, substances to prevent foaming, thickening agents, emulsifiers, fats, oils, waxes, organic solvents, bactericides, perfumes, dyes or pigments whose object is to colour the hair or the cosmetic or dermatological preparation itself, electrolytes and substances to combat greasiness of the hair are used.

The term “electrolytes within the meaning of the present invention” means water-soluble alkali-, ammonium-, alkaline earth- (including magnesium) and zinc salts of inorganic anions and any mixtures of such salts, of which it must be guaranteed that these salts are completely harmless pharmaceutically and cosmetically.

The anions according to the invention are preferably chosen from the group consisting of the chlorides, sulphates and hydrogen sulphates, phosphates, hydrogen phosphates and linear and cyclic oligophosphates as well as carbonates and hydrogen carbonates.

Cosmetic preparations which represent a shampooing agent preferably contain at least one anionic, non-ionic or amphoteric surfactant substance, or also mixtures of such substances in an aqueous medium and auxiliary agents, such as those normally used for this purpose. The surfactant substance and/or mixtures of these substances can be present in a concentration of between 1 wt. % and 50 wt. % in the shampooing agent.

A cosmetic preparation in the form of a lotion which is not rinsed out, in particular a lotion for setting the hair, a lotion which is used whilst drying the hair, a styling and treatment lotion, generally represents an aqueous, alcoholic or aqueous-alcoholic solution, and also contains e.g. comb polymers.

The compositions according to the invention optionally contain the additions usual in cosmetics, for example perfume, thickener, dyes, deodorants, antimicrobial substances, degreasing agents, complexing and sequestering agents, pearl shine agents, plant extracts, vitamins, active substances and the like.

All quantities indicated, proportions and percentages, unless otherwise specified, relate to the weight and total quantity or to the total weight of the preparations.

The following examples are intended to clarify the present invention, without limiting it. Weight percentages are indicated.

In the following examples CLA1 signifies the following fatty acid CLA isomer preparation:

TABLE 1
CLA 1

| Fatty acid | wt. % |
|---|---|
| 16:0 | 6.9 |
| 18:0 | 2.5 |
| 18:1 | 15.3 |
| 18:2 | 0.8 |
| 18:2(CLA) | 73.8 a) |
| (remainder not defined) | |

a) CLA composition (-octadiene acid)

| | |
|---|---|
| 9c, 11t/9t, 11c- | 34.6 |
| 10t, 12c- | 35.9 |
| 9c, 11c/10c, 12c- | 1.7 |
| 9t, 11t/10t, 12t- | 1.6 |

### EXAMPLES 1-3

Conditioner shampoo with pearl-shine

| | 1 | 2 | 3 |
|---|---|---|---|
| Polyquaternium-10 | 0.5 | 0.5 | 0.5 |
| Sodium laureth sulphate | 9.0 | 9.0 | 9.0 |
| Cocamidopropyl betain | 2.5 | 2.5 | 2.5 |
| Pearl shine agent | 2.0 | 2.0 | 2.0 |
| Coenzyme Q10 | 0.3 | 0.03 | 3.5 |
| Carnitine | — | 0.5 | — |
| CLA1 | — | — | 0.3 |
| Preservative, perfume, thickener, | q.s. | q.s. | q.s. |
| pH-adjustment agent and | | | |
| dissolving intermediary | | | |
| Water, completely salt-free | ad 100.0 | ad 100.0 | ad 100.0 |

The pH is adjusted to 6.

### EXAMPLES 4-6

Clear conditioner shampoo

| | 4 | 5 | 6 |
|---|---|---|---|
| Polyquaternium-10 | 0.5 | 0.5 | 0.5 |
| Sodium laureth sulphate | 9.0 | 9.0 | 9.0 |
| Cocamidopropyl betain | 2.5 | 2.5 | 2.5 |
| Coenzyme Q10 | 0.1 | 0.4 | 0.2 |
| Minoxidil | 7.0 | 7.0 | 7.0 |
| Lipoic acid | 0.2 | — | — |
| Folic acid | — | 0.2 | — |
| Arginine | — | — | 1.0 |
| Preservative, perfume, thickener, | q.s. | q.s. | q.s. |
| pH-adjustment agent and | | | |
| dissolving intermediary | | | |
| Water, completely salt-free | ad 100.0 | ad 100.0 | ad 100.0 |

The pH is adjusted to 6.

### EXAMPLES 7-9

Clear light shampoo with volume effect

| | 7 | 8 | 9 |
|---|---|---|---|
| Sodium laureth sulphate | 10.0 | 10.0 | 10.0 |
| Cocamidopropyl betain | 2.5 | 2.5 | 2.5 |
| Coenzyme Q10 | 0.8 | 0.5 | 0.4 |
| Finasteride | 1.0 | 1.0 | 1.0 |
| Acetyl carnitine | 1.0 | 0.1 | — |
| Disodium succinate | — | 1.0 | — |
| Lipoic acid | — | — | 0.5 |
| Carnitine | — | — | 0.1 |
| Arginine | — | — | 0.2 |
| Preservative, perfume, thickener, | q.s. | q.s. | q.s. |
| pH-adjustment agent and | | | |
| dissolving intermediary | | | |
| Water, completely salt-free | ad 100.0 | ad 100.0 | ad 100.0 |

The pH is adjusted to 5.5.

### EXAMPLES 10-13

Hairspray

| | 10 | 11 |
|---|---|---|
| Octyl acrylamide/acrylates/butyl | 2.5 | 2.5 |
| amino ethyl methacrylate copolymer | | |
| Coenzyme Q10 | 0.05 | 0.08 |
| Gamma linoleic acid | 8.0 | 8.0 |
| 1-(4′-tert.butylphenyl)-3-(4′methoxy- | 1.0 | — |
| phenyl)propane-1,3-dione (Parsol 1789) | | |
| 2,4,6-trianilino-(-p-carbo-2′-ethyl-1′-hexyloxy)- | — | 1.0 |
| 1,3,5,-triazine | | |
| Abs. ethanol | 39.0 | 39.0 |
| Perfume, dissolving intermediary, | q.s. | q.s. |
| neutralisation agent/pH adjustment agent, | | |
| care products | | |
| Dimethyl ether | ad 100 | ad 100 |
| | 12 | 13 |
| PVP/VA copolymer | 8.0 | 8.0 |
| Coenzyme Q10 | 0.01 | 0.05 |
| Zinc chloride | 6.0 | 6.0 |
| 3-(4-methyl benzylidene)-camphor | 1.0 | — |
| 4-methoxy cinnamic acid-(2-ethyl hexyl)-ester | — | 1.0 |
| Abs. ethanol | 39.0 | 39.0 |
| Perfume, dissolving intermediary, | q.s. | q.s. |
| care products | | |
| Dimethyl ether | ad 100 | ad 100 |

### EXAMPLES 14-16

Hair treatment

| | 14 | 15 | 16 |
|---|---|---|---|
| Hydroxypropyl methyl cellulose | 0.5 | 0.5 | 0.5 |
| Cetrimonium bromide | 1.0 | 1.0 | 1.0 |
| Glycerine | 3.0 | 3.0 | 3.0 |
| Cetearyl alcohol | 2.5 | 2.5 | 2.5 |
| Glyceryl stearate | 2.0 | 2.0 | 2.0 |
| Ubiquinone Q10 | 0.02 | 0.0002 | 0.2 |
| Epicatechin | 4.0 | 4.0 | 4.0 |
| Carnitine | 2.0 | 0.4 | — |
| CLA1 | — | 0.5 | — |
| Lipoic acid | — | 0.3 | 1.0 |
| Alpha glucosyl rutin | — | 0.2 | — |
| Arginine | — | — | 1.5 |
| Preservatives, perfume, | q.s. | q.s. | q.s. |
| pH adjustment agent, | | | |
| Water, completely salt-free | ad 100.0 | ad 100.0 | ad 100.0 |

The pH value is adjusted to 3.5.

### EXAMPLES 17-19

Hair rinsing

| | 17 | 18 | 19 |
|---|---|---|---|
| Behentrimonium chloride | 1.0 | 1.0 | 1.0 |
| Glycerine | 3.0 | 3.0 | 3.0 |
| Hydroxyethyl cellulose | 0.2 | 0.2 | 0.2 |
| Cetearyl alcohol | 3.0 | 3.0 | 3.0 |
| Ubiquinone Q10 | 0.0004 | 0.05 | 0.5 |
| Epigallocatechin-3-gallate | 2.0 | 2.0 | 2.0 |
| Folic acid | 0.8 | — | — |
| Vitamin E | — | 0.2 | — |
| Disodium succinate | — | — | 1.0 |
| Preservatives, perfume | q.s. | q.s. | q.s. |
| pH adjustment, | | | |
| Water, completely salt-free | ad 100.0 | ad 100.0 | ad 100.0 |

The pH value is adjusted to 3.0.

### EXAMPLES 20 AND 21

Setting mousse

| | 20 | 21 |
|---|---|---|
| PVP/VA copolymer | 8.0 | 8.0 |
| Hydroxy ethyl cetyl dimonium phosphate | 0.1 | 0.1 |
| Coenzyme Q10 | 0.07 | 0.01 |
| Pinacidil | 2.0 | 2.0 |
| Carnitine | 0.1 | — |
| Arginine | — | 1.0 |
| Perfume, dissolving intermediary, | q.s. | q.s. |
| care products | | |
| Abs. ethanol | 10.0 | 10.0 |
| Propane/butane | 10.0 | 10.0 |
| Water, completely salt-free | ad 100.0 | ad 100.0 |

### EXAMPLES 22 AND 23

| | 22 | 23 |
|---|---|---|
| PVP/VA copolymer | 5.0 | 5.0 |
| Polyquaternium-16 | 2.0 | 2.0 |
| Hydroxy ethyl cetyl dimonium phosphate | 0.1 | 0.1 |
| Coenzyme Q10 | 0.0001 | 0.004 |
| Minoxidil | 2.0 | 2.0 |
| Finasteride | 1.0 | 1.0 |
| Carnitine | — | 1.0 |
| Arg in i ne | — | 2.0 |
| Lipoic acid | 0.2 | 0.5 |
| CLA1 | — | 1.0 |
| 1-(4′tert.butyl phenyl)-3-(4′methoxy- | 1.0 | 2.0 |
| phenyl)propane-1,3-dione (Parsol 1789) | | |
| 2,4,6-trianilino-(-p-carbo-2′-ethyl-1′-hexyloxy)- | 1.0 | 2.0 |
| 1,3,5 triazine | | |
| Perfume, dissolving intermediary, | q.s. | q.s. |
| care products | | |
| Abs. ethanol | 10.0 | 10.0 |
| Propane/butane | 10.0 | 10.0 |
| Water, completely salt-free | ad 100.0 | ad 100.0 |

### EXAMPLES 24 AND 25

Styling gels

| | 24 | 25 |
|---|---|---|
| PVP/VA copolymer | 5.0 | 5.0 |
| Ceteareth-25 | 0.1 | 0.1 |
| Carbomers | 0.8 | 0.8 |
| Coenzyme Q10 | 0.01 | 0.001 |
| Minoxidil | 1.0 | 1.0 |
| Finasteride | 1.0 | 1.0 |
| Gamma linoleic acid | 4.0 | 4.0 |
| Acetyl carnitine | 0.2 | — |
| Alpha glucosyl rutin | 0.2 | — |
| CLA1 | — | 0.5 |
| Perfume, dissolving intermediary, care products | q.s. | q.s. |
| neutralisation agents/pH-adjustment agents | | |
| Abs. ethanol | 10.0 | 10.0 |
| Water, completely salt-free | ad 100.0 | ad 100.0 |

### EXAMPLE 26

W/O Cream

| | wt. % |
|---|---|
| Vaseline DAB 9 | 13.0 |
| Glycerine DAB 9 | 6.3 |
| Water, completely salt-free | 34.4 |
| Paraffin oil (Mineral oil 5E, Shell) | 43.2 |
| Cetearyl alcohol/PEG-40-Castor Oil/ | 2.5 |
| Sodium cetearyl sulphate (Emulgade F, | |
| Henkel KGaA) | |
| Coenzyme Q10 | 0.6 |
| Minoxidil | 2.0 |

0.6 parts coenzyme Q₁₀ dissolved in 3 parts paraffin oil are worked into the warm fat phase at 75° C. The fat phase is then added to the warm water fat phase at 75° C., stirred and homogenised, until a homogeneous, light yellow cream is formed.

### EXAMPLE 27

W/O Cream

| | wt. % |
|---|---|
| PEG-1 glyceryl-oleostearate+ | 8.0 |
| paraffin wax | |
| Vaseline DAB | 2.8 |
| Paraffin wax/paraffin | 1.8 |
| Paraffin oil (Mineral oil 5E, Shell) | 11.5 |
| Ceresin | 2.2 |
| Octyl dodecanol | 10.0 |
| Coenzyme Q10 | 0.8 |
| Finasteride | 1.0 |
| Propylene glycol | 1.0 |
| Glycerine | 1.0 |
| Carnitine | 0.7 |
| Water, completely salt-free | 59.4 |
| Total additives (perfume, | 0.8 |
| preservatives, stabilisers) | |

0.8 parts coenzyme Q₁₀ dissolved in 6 parts paraffin oil are worked into the warm fat phase at 75° C. The fat phase is then added to the warm water fat phase at 75° C., stirred and homogenised, until a homogeneous, light yellow cream is formed.

### EXAMPLE 28

O/W Cream

| | wt. % |
|---|---|
| Octyl dodecanol | 9.3 |
| (Eutanol G, Henkel KGaA) | |
| Cetearyl alcohol/PEG-40-Castor Oil/ | 3.7 |
| Sodium cetearyl sulphate (Emulgade F, | |
| Henkel KGaA) | |
| Water, completely salt-free | 72.7 |
| Glycerine DAB 9 | 4.6 |
| Paraffin oil (Mineral oil 5E Shell) | 7.7 |
| Coenzyme Q10 | 0.9 |
| Minoxidil | 1.0 |
| Finasteride | 1.0 |
| Gamma linoleic acid | 1.0 |
| Epicatechin-3-gallate | 2.0 |
| Arginine | 1.0 |

0.9 parts coenzyme Q₁₀ dissolved in 4 parts paraffin oil are worked into the warm fat phase at 75° C. The fat phase is then added to the warm water fat phase at 75° C., stirred and homogenised, until a homogeneous, light yellow cream is formed.

### EXAMPLE 29

O/W Lotion

| | wt. % |
|---|---|
| Steareth-2 | 3.0 |
| Steareth-21 | 2.0 |
| Cetearyl alcohol/PEG-40-Castor Oil/ | 2.5 |
| Sodium cetearyl sulphate (Emulgade F, | |
| Henkel KGaA) | |
| Paraffin oil (Mineral oil 5E Shell) | 14.4 |
| Propylene glycol | 1.0 |
| Coenzyme Q10 | 0.1 |
| Minoxidil | 2.0 |
| Folio acid | 0.9 |
| Glycerine | 1.0 |
| Water, completely salt-free | 74.3 |
| Total additives (perfume, | 0.8 |
| preservative, stabiliser) | |

0.1 parts coenzyme Q₁₀ dissolved in 5.2 parts paraffin oil are worked into the warm fat phase at 75° C. The fat phase is then added to the warm water fat phase at 75° C., stirred and homogenised, until a homogeneous, light yellow lotion is formed.

### EXAMPLE 30

O/W Lotion

| | wt. % |
|---|---|
| Octyl dodecanol | 5.6 |
| (Eutanol G, Henkel KGaA) | |
| Cetearyl alcohol/PEG-40-castor oil/ | 8.9 |
| sodium cetearyl sulphate (Emulgade F, | |
| Henkel KGaA) | |
| Cetearyl isononanoate | 7.5 |
| (Cetiol 5N, Henkel KGaA) | |
| Water, completely salt-free | 62.3 |
| Glycerine DAB 9 | 4.7 |
| Paraffin oil (Mineral oil 5E Shell) | 10.0 |
| Coenzyme Q10 | 0.4 |
| Zinc chloride | 8.0 |
| Disodium succinate | 0.6 |

0.4 parts coenzyme Q₁₀ dissolved in 6 parts paraffin oil are worked into the warm fat phase at 75° C. The fat phase is then added to the warm water fat phase at 75° C., stirred and homogenised, until a homogeneous, light yellow lotion is formed.

### EXAMPLE 31

Oil

| | parts by weight |
|---|---|
| Glyceryl tricaprylate | 21.0 |
| (Miglycol 812, Dynamit Nobel) | |
| Hexyl laurate | 20.0 |
| (Cetiol A, Henkel KGaA) | |
| Octyl stearate | 20.0 |
| (Cetiol 886, Henkel KGaA) | |
| Paraffin oil | 35.0 |
| (Mineral oil 5E Shell) | |
| CLA1 | 2.0 |
| Coenzyme Q9 | 1.6 |
| Coenzyme Q10 | 0.4 |
| Minoxidil | 1.0 |
| Gamma linoleic acid | 8.0 |

The components are stirred at 25° C., until a homogeneous, clear mixture is formed.

### EXAMPLE 32

Hair lotion

| | wt. % |
|---|---|
| Minoxidil | 2.0 |
| Coenzyme Q10 | 1.0 |
| Ethanol | 10.0 |
| Water | 89.0 |

The components are mixed and dissolved.

1. Use
a) of one or more compounds from the group of bioquinones in combination with
b) one or more compounds from the group of potassium channel openers and in combination with
c) one or more compounds from the group of 5-alpha-reductase inhibitors,
for the production of cosmetic or dermatological preparations for treating and preventing seborrhoeic symptoms.

2. Use according to claim 1 for the production of cosmetic or dermatological preparations for the treatment and prevention of dandruff.

3. Use according to claims 1 and 2, characterised in that one or more compounds from the group consisting of carnitine, arginine, succinic acid, folic acid, conjugated fatty acids and their respective derivatives, and antioxidants are additionally used.

4. Use according to claims 1 to 3, characterised in that the following combinations are used:
a) ubiquinones, in particular coenzyme Q-10, and minoxidil, or
b) ubiquinones, in particular coenzyme Q-10, and finasteride and/or gamma linoleic acid, or
c) ubiquinones, in particular coenzyme Q-10, and minoxidil, and finasteride and/or gamma linoleic acid.

5. Cosmetic or dermatological preparation, characterised in that it contains a combination
a) of one or more compounds from the group of bioquinones with
b) one or more compounds from the group of potassium channel openers and with
c) one or more compounds from group of 5-alpha-reductase inhibitors.

6. Preparation according to claim 5, characterised in that it additionally contains one or more compounds from the group consisting of carnitine, arginine, succinic acid, folic acid, conjugated fatty acids and their respective derivatives, and antioxidants.
